(19) United States
(12) Patent Application Publication
(10) Pub. No.: US 2004/0254646 A1
(43) Pub. Date: Dec. 16, 2004

(54) Provisional coupling mechanism

(76) Inventors: Kevin T. Stone, Winona Lake, IN (US); John R. White, Winona Lake, IN (US)

Correspondence Address: Stephen J. Foss, Harness, Dickey & Pierce, P.L.C., P.O. Box 828, Bloomfield Hills, MI, 48303, US

(21) Appl. No.: 10/462,577

(22) Filed: Jun. 16, 2003

(51) Int. Cl. A61F 2/36

(52) U.S. Cl. 623/23.15

(57) ABSTRACT

An implant for the anatomy, which includes a connecting portion that can be used during a secondary or revisionary procedure to interconnect the first implant with a second anatomical implant. For example, an elongated shaft may be implanted into a femur to reinforce the femur. In addition, the elongated implant includes a connecting portion, which is covered by a temporary or provisional cap. If a revision or secondary procedure is necessary, the provisional cap may be removed to expose an interconnecting portion to receive a secondary implant during the revisionary procedure.

### FIELD

The present invention relates to prosthetic implants; and particularly to modular prosthetic implants, including provisional coupling portions.

### BACKGROUND

It is generally known to use various artificial implants to replace diseased or injured portions of an anatomy. A biocompatible rod can be implanted inside of a long bone to reinforce or stabilize the long bone, which may be injured or otherwise weakened. For example, the femur may be irreparably fractured or weakened due to disease or injury and require substantial reconstruction. In the reconstruction, an elongated rod may be inserted into the femur to re-enforce the femur to help rehabilitate a patient. The elongated member can be inserted into the femur to help strengthen and enhance the femur for physical use by the recipient after the procedure.

When such extreme measures are taken to repair a long bone, such as the femur, additional injuries may occur due to the already weakened nature of the bony structure. After additional use or injury, the head of the femur or the condyles of the knee may need to be replaced or otherwise repaired. However, due to the previous placement of an elongated member in the femur, it may be difficult to implant the appropriate knee or hip replacements. For example, to insert a hip replacement, the femur is resected to remove the head portion of the femur and a prosthetic head is implanted to replace the head of the femur. Generally, the head implant includes an elongated stem, which is implanted into the femur. If the elongated member is already implanted in the femur, however, it may be difficult to properly place the head implant in the femur. It may be required to remove the previously implanted elongated member and replace it with an exceptionally elongated stem portion for the head replacement.

Additional or revision implantations may become more complicated after the implant of elongated members in particular bony structures. Either the previously implanted portions may need to be removed or extraordinary fixation measures may be necessary to affix the additional implant member.

Therefore, it is desirable to provide a system that allows for implantation of a first implant and implantation of a second implant without disturbing the first implant. It is also desired to provide a system that will allow implantation of two implants that can be fixed together to provide a desired result.

### SUMMARY

A prosthetic implant that includes provisional attaching sections for attachment of later or secondary implants, if the later implants are necessary. Generally, an elongated rod implant may be used to reinforce a long bone, such as the femur. The elongated member includes, on at least one end, a section which may receive and interconnect with a second implant if the second implant is necessary.

For example, the elongated member may be placed within the femur. At a later date, the femur may incur a second injury, such as a fracture of the femoral head. The femur may then be resected and a femoral head prosthetic implanted onto the femur such that a portion of the femoral head prosthetic interconnects with the elongated member at the provisional attaching section. This allows the elongated member to remain in place and facilitate the implantation of the prosthetic femoral head. It will be understood that various prosthetics may also include elongated portions, such as the stem of a femoral head prosthetic, which include a provisional attaching section to receive a second implant such as a knee implant.

According to an embodiment an implant for a portion of an anatomy includes a member having a first end and a second end. A provisional attachment section is positioned at one of the first end or the second end. The member is implanted into the anatomy and the first or second end is substantially covered by a bony portion in a first selected position. The provisional attachment section may receive a secondary implant during a revision surgery.

According to an alternative embodiment a prosthetic for implanting into an anatomy includes a member having a first end and a second end. A provisional connecting section is included at one of the first end or the second end. A prosthetic portion, to replace an articulating portion of the anatomy, has a receiving connection to interconnect with the provisional connecting section. The member is implanted into a bony portion and receives the prosthetic.

According to an additional alternative embodiment a prosthetic includes a prosthetic portion to replace a first portion of bone. The prosthetic portion has a connecting section. A member to replace a second of the bony portions includes a provisional connecting portion to interconnect with the connecting section. The prosthetic portion, member, or a combination are implanted to replace selected bony portions at selected times such that the prosthetic portion and the member interconnect at implantation.

An alternative embodiment includes a prosthetic for implantation into an anatomy including a first prosthetic member to articulate with a first bony portion. An elongated member to reinforce a second bony portion includes at least one provisional attachment portion to interconnect with the first prosthetic member. A second prosthetic member has a connecting portion to interconnect with the provisional attachment portion at a selected time. The elongated member is first implanted to allow interconnection with the provisional attachment portion at a selected time.

According to an alternative embodiment is a method of implanting an elongated member into a first bony portion including a provisional attachment section such that a prosthetic selectively interconnects with the provisional attachment section. The method includes preparing a first bony portion for implantation of the elongated member. The elongated member is implanted into the prepared bony portion. The provisional attachment section is preserved after implanting the elongated member. A revision procedure is performed to expose the provisional attachment section. The prosthetic is implanted to interconnect with the provisional attachment section.

Further areas of applicability will become apparent from the detailed description provided hereinafter. It should be understood that the detailed description and the various examples, while indicating the various embodiments, are intended for purposes of illustration only and are not intended to limit the scope of the following claims.

### BRIEF DESCRIPTION OF THE DRAWINGS

The present invention will become more fully understood from the detailed description and the accompanying drawings, wherein:

FIG. 1 is an exploded view of an elongated implant, including a provisional attachment end according to an embodiment of the present invention;

FIG. 2 illustrates a plurality of secondary implants that may be affixed to one of the provisional attachment ends of an implant, including a provisional attachment portion;

FIGS. 3A-3C illustrate a method of using an elongated implant, including a provisional attachment portion and an implant;

FIG. 4A illustrates an alternative embodiment, including an elongated member and an anatomical implant;

FIG. 4B-4C illustrates a method of using the alternative embodiment, including an elongated member and an anatomical implant;

FIGS. 5A-5C illustrate an elongated implant, according to an embodiment, including a provisional attachment portion; and

FIGS. 6A-6D illustrate an elongated implant, including a provisional attachment portion according to a variety of embodiments.

### DETAILED DESCRIPTION OF THE VARIOUS EMBODIMENTS

The following description of various embodiments is merely exemplary in nature and is in no way intended to limit the scope of the claims, its application, or uses. Although the following discussion relates specifically to elongated implants, including provisional attachment portions for implant into long bones, such as the femur or the humerus, it will be understood that the elongated implant can be implanted into any number of portions of the anatomy. In addition, any number of secondary implants can be affixed to the elongated implant for fixation in the anatomy.

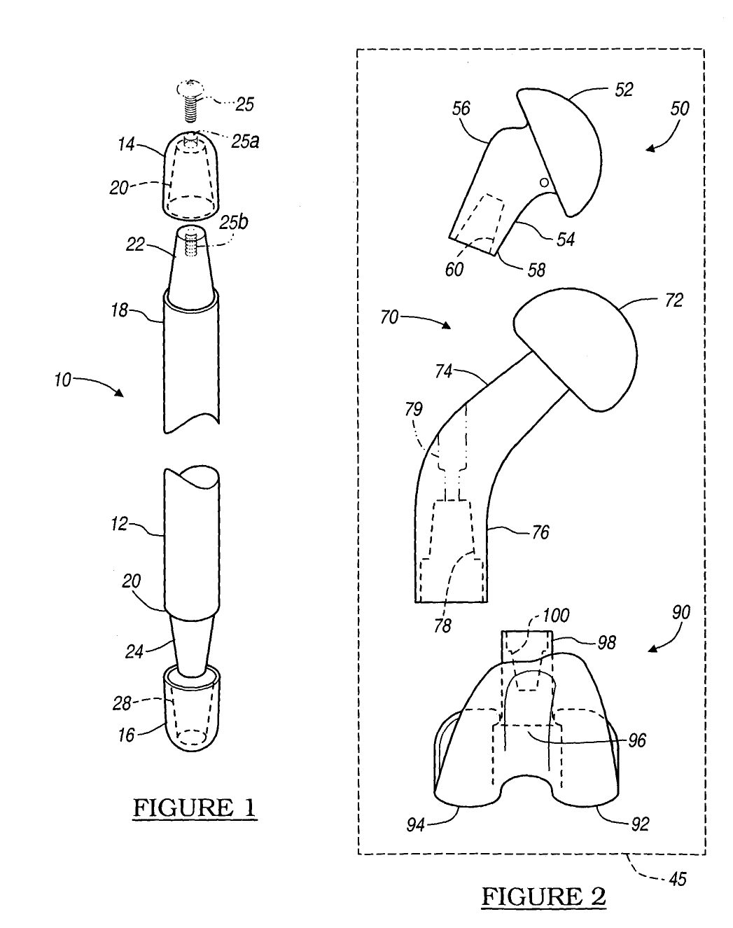

With reference to FIG. 1, an elongated implant member 10 includes an elongated shaft 12 and a plurality of caps 14 and 16. Formed at provisional attachment ends 18 and 20 of the elongated shaft 12 are provisional attachment portions or section 22 and 24. The provisional attachment portions 22 and 24 allow the respective caps 14 and 16 to be affixed to the elongated shaft 12 for a selected time. In addition, and described further herein, the provisional attachment portions 22 and 24 allow various other implants to be affixed to the elongated shaft 12. The provisional attachment portions 22 and 24 may define a male taper. The male tapers of the provisional attachment portions 22 and 24 are received within respective female tapers 26 and 28 of the respective caps 14 and 16. The male tapers of the provisional attachment portions 22 and 24 form a Morse taper fit with the female tapers 26 and 28. In this way, the caps 14 and 16 are affixed to the elongated shaft 12 for the selected time. Various other appropriate methods may be used to form the provisional or attachment portions 22 and 24. In addition to the Morse taper a locking screw 25 (shown in phantom) may be provided to be received through a cap bore 25a and into a stem bore 25b to lock the cap 14 to the elongated implant member 10. It will be understood a similar screw may be provided to lock the cap 16 to the member 10 as well.

It will be understood that the elongated shaft 12 may include a plurality of provisional attachment portions. For example, the elongated shaft 12 may be a modular shaft that includes a provisional attachment portion fixed at a intermediate point between the two ends 18 and 20 of the elongated shaft 12. In this way, the elongated shaft 12 can be selectively implanted and include more than one portion to allow for easy selection of a specific size. This allows a physician to form the appropriate size implant for a patient intra-operatively, rather than requiring pre-operative determination of the appropriate length of the elongated shaft 12. In addition, the modular shaft may allow specific portions of the shafts to be removed to allow for easy implantation of a waiter implant.

With reference to FIG. 2, a plurality of secondary implants 45 may include, for example, a humeral prosthesis 50, a femoral prosthesis 70 and a knee prosthesis 90. It will be understood that the secondary implant 45 may be any appropriate implant, where a humeral, femoral, and knee prosthesis are merely exemplary. Further examples, also not limiting, include a wrist, an ankle, an elbow, or other appropriate implants. Each of the secondary implants 45 may also include a locking bore 79 to receive the locking screw 25 to firmly affix the secondary implant 45 to the member 10. The humeral prosthesis 50 includes a head portion 52, which is adapted to articulate with a shoulder portion of the anatomy after implantation. Extending from the head 52 is a stem 54. The stem 54 is implanted into the humerus to replace the articulating portion of the humerus due to injury or disease. The stem 54 includes a proximal end 56 and a distal end 58. The distal end 58 includes a receiving connection portion 60 that can interconnect at the provisional connection portion 22 or 24 of the elongated implant 10. Therefore, the implant 10 may remain in the humerus, if previously implanted, and the humeral implant 50 may interconnect through the connections of the receiving connection 60 and the provisional connection 22.

With additional reference to FIG. 2, the femoral implant 70 includes a head portion 72, a neck portion 74, and a stem 76. Formed in a distal end of the stem 76 is a provisional connection receiving portion 78. Similar to the humeral implant 50, the provisional connection receiving portion 78 can interconnect with the provisional connection 22 or 24 of the implant 10. The stem portion 76 of the femoral implant 70 may be any appropriate length necessary to interconnect with the elongated implant 10 and be properly fixed within the femur. Furthermore, other fixation means, such as bone cement or bone in-growth texture, may be used in conjunction with the femoral implant 70.

With continuing reference to FIG. 2, a knee implant 90 includes a first condyle 92, a second condyle 94, and a patella groove 96. These portions of the knee implant 90 are generally integrally formed and an implant stem 98 extends from these portions to be received in the femur. On a proximal end of the implant stem 98 is a provisional connecting portion 100. As described above, the provisional connection portion 100 may receive the provisional connection portion 24 of the implant 10. Therefore, if the implant 10 has been previously implanted in the femur, either the femoral implant 70 or the knee implant 90 may be implanted into the femur and engage the implant 10 to create a substantially permanent connection between the implant portions within the femur.

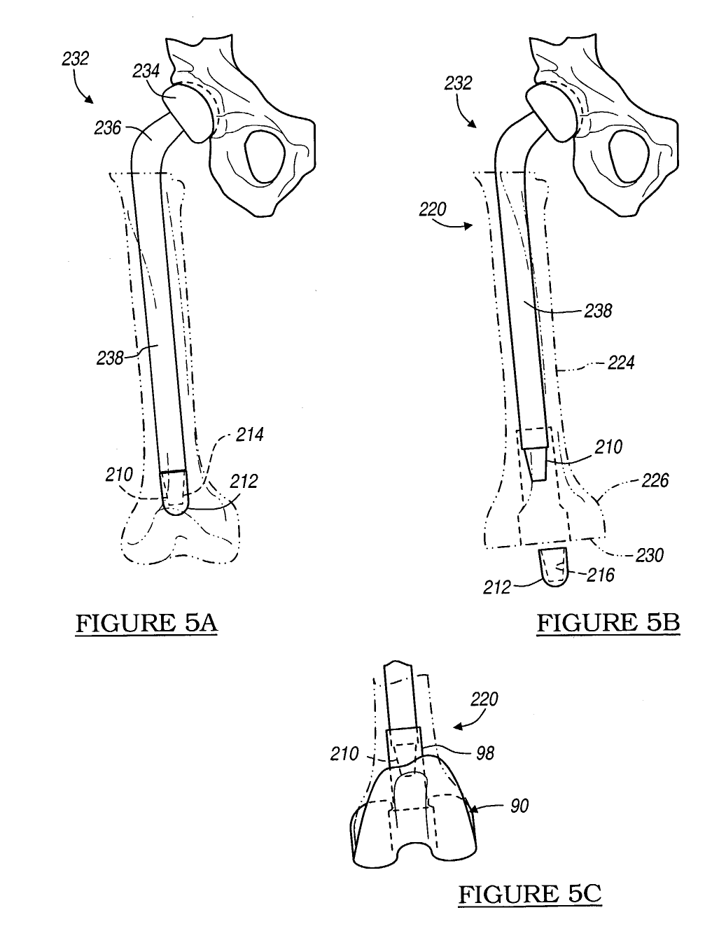

It will be understood that the various provisional connection portions in various provisional connection receiving portions may be formed as any complimentary interconnection portions, illustrated in FIG. 5. As described further herein, specific threads, locking rings, and Morse taper connections may be used to provide the appropriate connection for the various portions. Simply, the type of interconnection may be selected depending upon the particular implant, patient condition, or physician preference. Moreover, although a humeral implant, femoral implant, and knee implant are illustrated, various other implants may be used in conjunction with various other implants. Generally, the first implant, such as the implant 10, is implanted into a long bone, which includes provisional connecting portions 22 and 24. During a revision or after additional surgery, a second implant may be implanted to interconnect with the first implant 10 and include a connection portion to interconnect with the first implant.

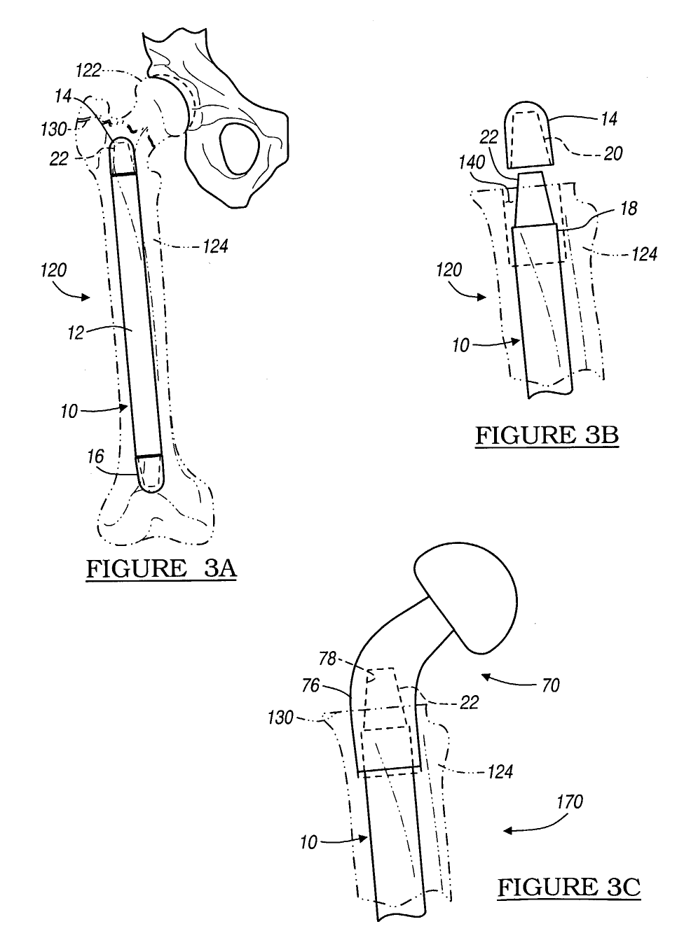

With reference to FIGS. 3A-3C, a method of using the various implants with provisional connections is illustrated and described. It will be understood that although the following description relates specifically to a femur and femoral implants that the implants with provisional connections may be used in numerous portions of the anatomy.

With initial reference to FIG. 3A, a femur 120 includes a head portion 122 and a shaft portion 124. As illustrated here, the shaft portion includes the implant 10, which includes the elongated shaft 12, the proximal provisional connection portion 22 with the cap 14 installed thereon. The elongated implant 10 may have been implanted into the femur 120 for any number of reasons. For example, the elongated implant 10 may have been implanted to strengthen the shaft of the femur 120 or to re-enforce the femur 120 after removing bone marrow due to various diseases, such as cancer. If the femur is broken in a portion of the femoral shaft 124, which does not require replacement of either the knee portion or the humeral head 122, the elongated shaft 10 may be inserted into the two portions of the femur 120 and the femur 120 sealed around the elongated implant 10.

Regardless, the reason the implant 10 includes provisional attachments that are buried in the femur 120, or any other appropriate bone. The provisional attachment will be generally understood to be a connection that can be used during a revision surgery. For example, during a primary procedure the implant 10 may be implanted to strengthen the femur 120. The provisional attachments are covered with the caps 14, 16 and buried in the femur 120. These provisional attachments may be used in a revision procedure, for example, to attach a femoral head to the implant 10. It will be understood the provisional attachments do not receive an implant during a primary surgery or procedure, but generally during a revision procedure.

Specifically, the femur 120 may be reamed to appropriate dimensions to receive the elongated implant 10. Additional fixation means may then be used to ensure a proper fixation of the elongated implant 10 into the femur 120. For example, the exterior surface of the elongated implant 10 may include bone in-growth portions to receive bone in-growth or to allow for fixation using bone cement to ensure that the femur 120 is properly affixed to the elongated implant 10. In addition, the appropriate caps 14 and 16 are fixed to the provisional connection portions 22 and 24 to keep the provisional connection portions 22 and 24 substantially free of bone in-growth, bone fixation cement, biological fluid, or other undesired materials. This will allow for an easy access to the provisional connection portions 22 and 24 if such a secondary or revision surgery is required. Nevertheless, the elongated implant 10 generally extends a substantial length of the femur 120 from the proximal end to its distal end. Therefore, a majority of the femur's 120 length is covered by the elongated implant 10.

The femur 120, however, may later develop a fracture 130. The fracture may be a complete fracture, which will not heal on its own and requires a second implant, such as the femoral implant 70. To implant the femoral implant 70, the head portion 120 must be resected. It is generally known in the art to remove the head portion 122 and resect necessary portions of the femur 120 to provide for an implantation of the femoral implant 70.

With reference to FIG. 3B, the femur 120 has been resected to allow for the implantation of the femoral implant 70. In addition, the femoral shaft 124 has been resected in appropriate portion to receive the femoral implant 70. Moreover, the proximal portion 18 of the elongated implant 10 is exposed so that the proximal provisional connecting portion 22 is exposed. In addition, the cap 14 has been removed to expose the provisional connecting portion 22. It will be understood that it may be necessary to resect or ream portions around the periphery of the elongated implant 10. Specifically, it may be desirable to engage an extended portion of the elongated implant 10 with the femoral implant 70. Therefore, portions of the femoral shaft 124 may be reamed to form a femoral implant pocket 140. Nevertheless, it is understood that various methods of reaming and forming the femoral shaft 124 are generally known in the art and not specifically described here. In addition, physician preference may determine how much and where the resection occurs.

With reference to FIG. 3C, the elongated implant 10 has been interconnected with the femoral implant 70 during the revision or second implant procedure. Specifically, the provisional connection portion 22 of the elongated implant 10 has received the provisional receiving portion 78 of the femoral implant 70. Therefore, the elongated implant 10 need not be removed from the femur 120 to allow for the implant of the femoral implant 70. Simply, the femoral implant 70 includes the provisional connection portion 78 that can be received on the provisional connecting portion 22 of the elongated implant 10. In addition to this interconnection, other portions, such as a bone plug or bone cement 150 may be used to ensure proper fixation of the femoral implant 70 onto the femur 120 for physiological use. Also, as discussed above, the stem 76 of the femoral implant 70 may extend a distance along the elongated implant 10 to ensure a proper fixation of the femoral implant 70 into the femur 120. It will be understood that the distance that the femoral implant 70 extends along the elongated implant 10 may depend upon the individual patient and the materials from which the implant are formed.

The elongated implant and the secondary implant, such as the humeral implant 50, the femoral implant 70, or the knee implant 90 may all be formed of similar or complimentary materials. Specifically, the implants may be formed of the same material, such as titanium or cobalt chromium molybdenum alloys. In addition, it will be understood that the elongated implant may be formed of a material different from the secondary implants.

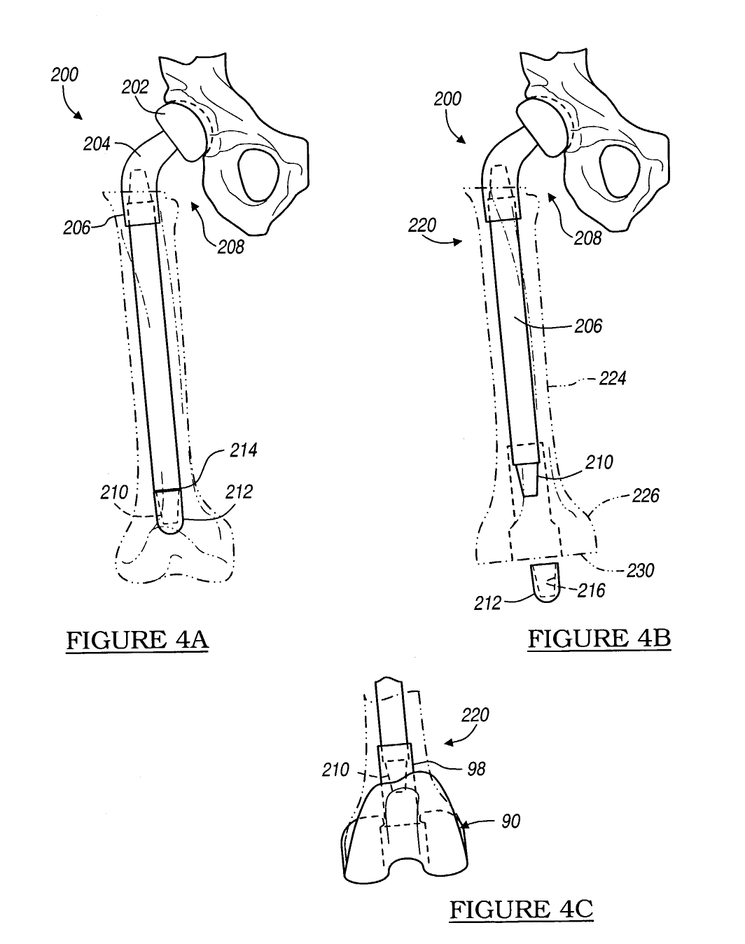

With reference to FIGS. 4A-4C, an implant 200 including a provisional attaching section 210 is illustrated. Specifically, a femoral and elongated implant 200 includes a head portion 202, a neck portion 204 and an elongated stem 206. The head portion 202 and the neck portion 204 define, and are similar to, other femoral portions and generally define a femoral portion 208. On a distal end of the elongated stem 206 is a provisional connecting portion 210. The provisional connecting portion 210 is covered by a provisional cap 212, which includes an internal receiving portion 214 to receive and cover the provisional connecting portion 210. Generally, the femoral portion 208 and the elongated stem 206 are similar to the elongated implant 10 being integrally formed with the femoral implant 70. Therefore, the elongated provisional implant 200 can be implanted into a long bone, such as the femur, to replace the head portion of the femur with the femoral portion 208 and strengthen the femur with the elongated stem 206.

With reference to FIG. 4B and continuing reference to FIG. 4A, the elongated femoral implant 200 can be implanted into a femur 220. The femoral portion 208 replaces the head portion of the femur 220. Therefore, the femoral implant portion 208 articulates with an acetabular portion, not particularly illustrated, in the anatomy. Furthermore, the elongated stem 206 can be used to reinforce the femoral shaft 224 of the femur 220. Furthermore, the elongated stem 206 can reach substantially to the distal end of the femur 220 and be implanted adjacent or near the knee 226 of the femur 220. At the distal end, the provisional connection portion 210 is covered by the provisional cap 212. It will be understood that the implant 200 can be fixed in the femur using any of the above described methods.

During additional physiological use of the femur 220, additional injuries may occur to the femur 220. Specifically, portions of the knee 226 may develop a fracture 230. The fracture 230 may not be able to heal without an additional implant. However, because the elongated shaft 206 extends a substantial portion into the knee 226 it is difficult to resect the elongated stem 206. Furthermore, the bony portion into which a knee implant may be implanted may already be removed because the implantation of the elongated stem 206.

With reference to FIG. 4C, however, the secondary knee implant 90, also illustrated in FIG. 2, may be implanted using the connecting portion 100. Resecting the knee 226 and the necessary portions of the femur 220 are generally known in the art and not restated here. Nevertheless, the knee portion 226 and appropriate portions of the femur 220 are resected and reamed to receive the knee implant 90. Therefore, the implantation stem 98 of the knee may be implanted into the femur 220 and fixed with appropriate means, such as bone cement. In addition, the provisional connecting portion 210 of the elongated stem 206 is received in the provisional receiving portion 100 of the knee 90. Therefore, the knee 90 can be implanted into the femur 220, which already includes the elongated stem 206. In addition, the elongated stem 206 provided previous support to the femur 220 and also included the integrally formed femoral portion 208. In this way, it is possible to retain certain bony portions of the femur 220 while replacing those parts that have the injury or damage.

Therefore, it will be understood that the elongated implant 10 may be implanted as a support portion or may be implanted, such as the elongated femoral implant 200. Specifically, the elongated portion 12 or 206 is used to support a long bone and includes provisional connecting portions 22, 24, and 210 to allow for the later implantation of a secondary implant. Nevertheless, appropriate cap portions can be used to protect the provisional connecting portions 22, 24, and 210 until such time that a secondary implant is necessary. It will also be understood that, although the elongated implant 200 is illustrated specifically and described with a femoral implant portion 208, that the knee implant 90 may be formed integrally with elongated implant 10. In this way, the femoral implant 70 may be affixed to an elongated stem of a knee implant. Alternatively, the humeral implant 50 may include an elongated stem to allow for a secondary implant of other portions.

With reference to FIGS. 5A-5C, an implant 232, similar to the implant 200, is illustrated. The implant 232 also includes the provisional attaching or connection section 210. The implant 232 is generally similar to the implant 200, illustrated in FIG. 4A. The implant 232, however, is not a substantially modular implant. Therefore, the implants 232 include an integrally formed femoral head prosthesis 234, a neck portion 236 extending therefrom, and an elongated stem 238 integrally formed with the neck portion 236. Therefore, rather than providing a substantially modular component, which includes the head portion 202 and the stem 206, such as the implant 200, the implant 232 is substantially integrally formed as a single piece.

Nevertheless, formed at an end, for example a distal end, of the elongated stem 238 of the implant 232 is the provisional attaching portion 210. Provided over the provisional attachment portion 210 is the provisional cap 212. The provisional cap 212 also defines the internal receiving portion 214 to substantially receive and protect the provisional connecting portion 210. Therefore, the cap 212 still protects the provisional connecting portion 210 such that it may be used during a revision procedure, as exemplary described herein.

The implant 232 may be used for any appropriate implant procedure. For example, the implant 232, including the elongated integrally formed stem 238, may be used for a hip revision procedure when a first prosthetic head implant is replaced or fixed. Particularly, the elongated stem 238 may be used when the femur 220 of the patient has degraded over time and must be reinforced with the elongated stem 238, and generally requiring a replacement of the first femoral head prosthesis (not illustrated). Nevertheless, the elongated stem 238 may include the provisional attaching portion 210 for attachment of a secondary implant during a further revision procedure.

With reference to FIG. 5B, at a selected time a revision procedure may occur which resects a distal portion of the femur 222 such as a portion of the knee 226. The knee portion 226 of the femur 220 may be resected along a fracture 230 which is formed in the knee portion. Furthermore, a bore may be formed to access the provisional connecting portion 210 of the implant 232. Therefore, the provisional cap 212 can be removed to access the provisional connecting portion 210.

With further reference to FIG. 5C, after the provisional cap 212 has been removed, the secondary implant, such as the knee implant 90, can interconnect with the provisional connecting portion 210. Therefore, the knee implant 90 is substantially integrally interconnected with the femoral head and elongated stem implant 232 that was previously implanted. Therefore, it will be understood that the elongated stem portion 238 need not necessarily be modular compared to a first or primary implant. Rather, the elongated portion including the provisional attachment portion 210 may either be a separate implant or may be formed integrally, as a single unit, with a another implant, such as a femoral head.

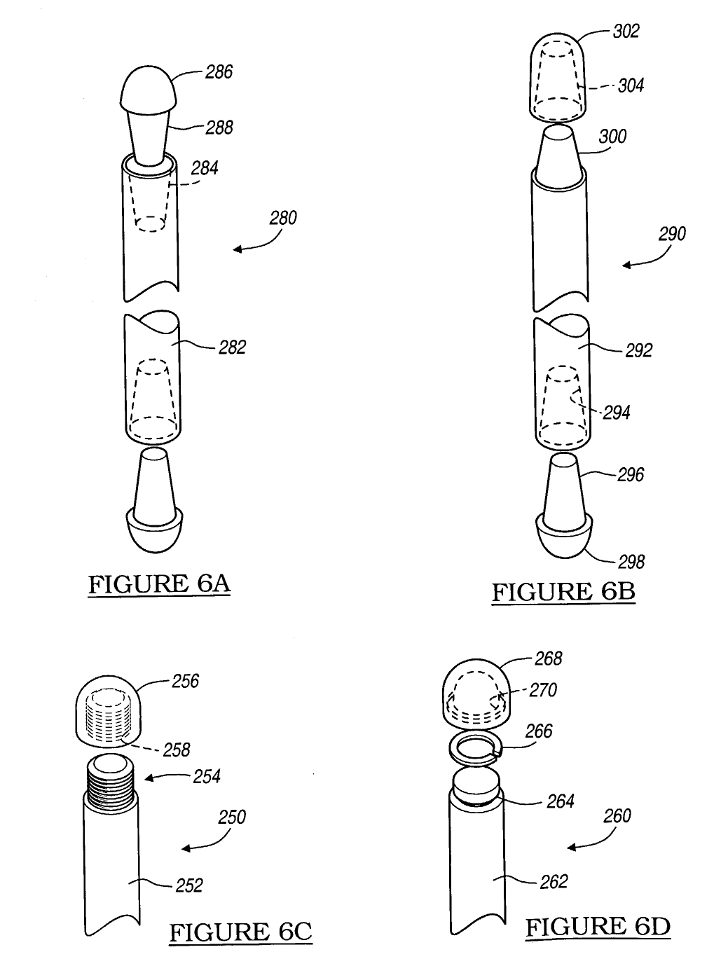

With reference to FIG. 6A-6D, a plurality of provisional connecting portions is illustrated. Specifically, an elongated implant 250, similar to the elongated implant 10, can include an elongated shaft 252 and a provisional connecting portion 254. Here, the provisional connecting portion 254 is an external thread. A provisional cap 256 includes an internal thread 258, which can thread with the external threads of the provisional connecting portion 254.

Alternatively, an elongated implant 260 includes an elongated shaft 262 and the provisional connecting portion 264. Specifically, the provisional connecting portion 264 may include a slot, which can receive a locking ring 266. The locking ring 266 is fit into a cap 268, which includes a complementary slot 270. Therefore, during the implantation of the cap 268 onto the elongated shaft 262, the locking ring 266 forms an interconnection between the cap 268 and the elongated shaft 262. Specifically, the locking ring 266 may be a split ring that flexes open and then snaps back into place to interconnect the cap 268 to the elongated shaft 262.

An additional alternative includes an elongated implant 282, which includes an elongated shaft 282. The elongated shaft defines a internal female taper 284, which defines a provisional connecting portion. A provisional cap 286 defines a male taper 288, which is receivable into the female taper 284. Therefore, rather than including the male taper on the elongated shaft 282, such as the elongated implant 10, the elongated shaft 282 defines a female taper to form a Morse taper fit with a male taper of a secondary implant.

A further alternative includes an implant 290. The implant 290 includes an elongated shaft 292. The elongated shaft 292 defines a first provisional connector 294, which defines a female taper 296. The female taper 296 is adapted to receive a male taper 296 of a provisional cap 298. On a proximal end of the elongated shaft 292 is a second provisional connector 300. The proximal provisional connector 300 generally defines a male taper. The male taper may be received in a provisional cap 302, which defines an internal female taper 304. Therefore, the implant 290 may include two different provisional connecting portions, such as the female taper 294 and the male taper 300. It will be understood that the implant 290 may include two different provisional connecting portions, including any number of a variety of provisional connecting portions, such as threads or various tapers. These specific and selected provisional connecting portions may be determined by a physician or the particular portion into which the elongated implants will be placed.

It will also be understood that although the above-described implants indicate that the elongated implant 10 is implanted first and the secondary implant is 45 are implanted during a revisionary implant, it will be understood that the opposite may also be possible. For example, a hip replacement may include the implant of the femoral implant 70. The femoral implant 70 including the provisional connecting portion 78 may be implanted into the femur to replace the femoral head. A suitable cap, not particularly illustrated, may also be included to cover the provisional connecting portion 78. During a revision surgery, the elongated implant 10 may then be implanted into the femur to reinforce the femur due to additional injury or disease. In this case, the provisional connecting portion 22 interconnects with the provisional connecting portion 78 of the femoral implant 70. Therefore, the femoral implant 70, including the femoral head 72, may be implanted first and later the elongated implant 10 implanted to interconnect with the femoral implant 70.

The description of the various embodiments is merely exemplary in nature and, thus, variations that do not depart from the gist and are intended to be within the scope of the invention. Such variations are not to be regarded as a departure from the spirit and scope.

What is claimed is:

1. An implant for a portion of an anatomy comprising:
a member including a first end and a second end; and
a provisional attachment section positioned at one of said first end or said second end;
wherein said member is implanted into the anatomy and said first or second end is substantially covered by a bony portion in a first selected position;
wherein said provisional attachment section is adapted to receive a secondary implant during a revision surgery.

2. The implant of claim 1, wherein said provisional attachment section includes a self-locking taper.

3. The implant of claim 2, wherein said provisional attachment portion is a male taper.

4. The implant of claim 1, wherein said member includes an elongated member wherein said first end and said second end are spaced a distance apart.

5. The implant of claim 1, wherein said member includes a rigid rod implantable to reinforce a bony structure of the anatomy.

6. The implant of claim 1, wherein said provisional attachment section includes at least one of a threaded section, a locking ring, a locking groove, and a female taper.

7. The implant of claim 1, wherein said member includes at least two provisional attachment sections.

8. The implant of claim 7, wherein said first end and said second end each define a provisional attachment section.

9. The implant of claim 1, further comprising a femoral head prosthetic, a humeral head prosthetic, or a knee prosthetic.

10. The implant of claim 1, wherein said member is integrally formed with at least one of a femoral head prosthetic, a knee prosthetic, or a humeral head prosthetic.

11. The implant of claim 1, wherein said first end includes a head portion of a femoral prosthetic and said second end includes a stem having said provisional attachment section.

12. The implant of claim 1, wherein said first end includes a humeral head portion and said second end includes a stem having said provisional attachment section.

13. The implant of claim 1, further comprising a cover to selectively cover said provisional attachment section, wherein said cover preserves said provisional attachment section after implantation.

14. A prosthetic for replacement of a portion of an anatomical structure during a revision procedure, comprising;
a prosthetic portion to replace a first portion of the anatomical structure;
a member to replace a second portion of the anatomical structure including a provisional connecting portion to interconnect with said connecting section; and
a cap to cover said provisional connecting portion during a primary procedure;
wherein said prosthetic portion, said member, or a combination are implanted to replace selected portions of the anatomical structure during the revision procedure such that said prosthetic portion and said member interconnect at implantation.

15. The prosthetic of claim 14, wherein said member includes a first end and a second end spaced apart, wherein at least one of said ends includes said provisional connecting portion.

16. The prosthetic of claim 14, wherein said prosthetic portion includes a femoral head prosthetic having a head portion and a stem portion, wherein said stem portion defines said connecting portion.

17. The prosthetic of claim 14, wherein said prosthetic portion includes a humeral head prosthetic having a head portion and a stem portion, wherein said stem portion defines said connecting section.

18. The prosthetic of claim 14, wherein said prosthetic portion includes a femoral head prosthetic having a head portion and a stem portion, wherein said stem portion defines said connecting section.

19. The prosthetic of claim 14, wherein said prosthetic portion includes a knee prosthetic having a condyle portion and a stem portion, wherein said stem portion defines said connecting section.

20. The prosthetic of claim 14, said prosthetic portion and said member form an integrated implant when said interconnection is made.

21. The prosthetic of claim 14, wherein said prosthetic portion and said member are formed integrally as one piece.

22. The prosthetic of claim 14, wherein said prosthetic portion and said member are modular and are connected during implantation.

23. A method of providing a first prosthetic, including a provisional connecting portion, in a first bony portion such that a second prosthetic selectively interconnects with the provisional attachment section during a revision procedure, the method comprising:
implanting the first prosthetic into the prepared bony portion;
preserving the provisional attachment section after implanting the elongated member;
performing a revision procedure to expose the provisional attachment section; and
implanting the second prosthetic to interconnect with the provisional attachment section.

24. The method of claim 23, further comprising;
affixing a cap to the provisional attachment section prior to implantation; and
implanting the first prosthetic such that the provisional attachment section is able to be accessed during the revision procedure.

25. The method of claim 23, further comprising;
providing an implant including the provisional attachment section including at least a taper, a threaded section, a locking ring, or a locking groove.

26. The method of claim 23, further comprising:
preparing a first bony portion including reaming a portion of the bony portion to receive the first prosthetic.

27. The method of claim 23, wherein preserving the provisional attachment section includes at least one of connecting a cover member to said first prosthetic and connecting a cover member to the provisional attachment section.

28. The method of claim 23, wherein performing a revision procedure includes removing a femoral head.

29. The method of claim 23, wherein performing a revision procedure includes removing a humeral head.

30. The method of claim 23, wherein performing a revision procedure includes removing a knee joint.

31. The method of claim 23, further comprising implanting the second prosthetic to replace a portion removed while performing the revision procedure.
